(19) United States
(12) Patent Application Publication
(10) Pub. No.: US 2023/0051694 A1
(43) Pub. Date: Feb. 16, 2023

(54) POLYPEPTIDE FRAGMENT B (MP-B) AND USE THEREOF

(71) Applicant: SHANGHAI LONGXIN BIOMEDICAL TECHNOLOGY CO., LTD., Shanghai (CN)

(72) Inventors: Caihua ZHANG, Shanghai (CN); Ying CHANG, Shanghai (CN)

(73) Assignee: SHANGHAI LONGXIN BIOMEDICAL TECHNOLOGY CO., LTD., Shanghai (CN)

(21) Appl. No.: 17/737,058

(22) Filed: May 5, 2022

Related U.S. Application Data
(63) Continuation of application No. PCT/CN2022/080880, filed on Mar. 15, 2022.

(30) Foreign Application Priority Data
Feb. 2, 2021 (CN) 202110145124.9

(51) Int. Cl. C07K 14/47; A61P 1/00; A23L 33/00; A23L 33/18

(52) U.S. Cl. CPC C07K 14/47; A61P 1/00; A23L 33/40; A23L 33/18; A61K 38/00

(57) ABSTRACT

A polypeptide fragment B (MP-B) has an amino acid sequence shown in SEQ ID NO: 1, in which an amino acid Xaa at position 10 is Arg, Phe, Glu, Thr, or absent, an amino acid Xaa at position 16 is Asn, Val, Leu, Gly, or absent, and an amino acid Xaa at position 25 is Ser, Glu, Met, Arg, or absent. The MP-B can significantly improve the colonic pathologic morphology and decrease a colonic histopathologic score and an expression level of colonic interferon-γ (IFN-γ) in an inflammatory bowel disease (IBD) mouse model, and shows the ability to interfere with the occurrence of IBD in mice.

### CROSS REFERENCE TO THE RELATED APPLICATIONS

This application is the national phase entry of International Application No. PCT/CN2022/080880, filed on Mar. 15, 2022, which is based upon and claims priority to Chinese Patent Application No. 202110145124.9, filed on Feb. 2, 2021, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBTF103-PKG_Sequence_Listing.txt, created on Apr. 12, 2022, and is 1,600 bytes in size.

### TECHNICAL FIELD

The present disclosure belongs to the technical field of biomedicine, and specifically relates to a polypeptide fragment B (MP-B) and a use thereof.

### BACKGROUND

Inflammatory bowel disease (IBD) is an idiopathic chronic intestinal inflammatory disease, and lesions thereof are mainly located in the colorectum, which involves the mucosa and muscularis mucosa, and even causes complications in the liver, gallbladder, muscle, skin, and coagulation function in severe cases. 20% to 30% of patients with recurring IBD may develop colorectal cancer (CRC). IBD is a very serious intestinal inflammatory disease, including the two categories of ulcerative colitis (UC) and Crohn's disease (CD). At present, it is believed that IBD is an intestinal inflammatory response caused by abnormal innate immunity and acquired immunity of the intestinal mucosa under the interaction of several factors such as environment, heredity, infection, and immunity, and an inflammatory response within the lamina propria of the intestinal mucosa is considered to be a cornerstone of the pathogenesis of IBD. In recent decades, there has been an increasing incidence of IBD. Traditional IBD treatment drugs, such as salicylic acids, steroid hormones, and immunosuppressants, effectively control the onset of IBD mainly by reducing the inflammation and regulating the immunologic disorder. However, these traditional methods cannot completely cure IBD, and often lead to some serious adverse reactions, causing severe hazard to the life quality of patients. Therefore, new IBD treatment methods are urgently needed.

In recent years, microecological preparations have gradually become a new idea for IBD treatment, and studies have shown that such preparations can improve various degrees of intestinal dysbacteriosis in IBD patients. Lactobacillus plantarum (L. plantarum) is a relatively common probiotic, and studies have shown that L. plantarum can inhibit the damage of pathogenic bacteria through adhesion and colonization in the intestine and regulate the intestinal permeability of immunodeficient mice, thereby interfering with the development of colitis. Micro integral membrane protein (MIMP) is an active polypeptide fragment isolated from the L. plantarum CGMCC 1258 strain that can compete with invasive pathogenic Escherichia coli (E. coli) to adhere to intestinal epithelial cells, which has a sequence shown in SEQ ID NO: 2 (THTVGSYFSVQNGYVGAFSQALGNSEYAMNSPLGSLDGRTTMYNLLGVKYLFAREDQ LKKQ), and can significantly improve an inflammatory state of the intestine and prevent the intestinal dysbacteriosis in IBD mice. However, MIMP is a biological macromolecule composed of 61 amino acids, and the large molecular weight is easy to cause immunogenicity and is not conducive to drug preparation, which limits its clinical practice. In addition, the large molecular weight is not conducive to the industrial production of drugs. From the perspective of medicinal value and economic benefits, MIMP is subjected to further structural modification and transformation to improve the pharmacological activity and/or druggability of the MIMP fragment, thereby facilitating the clinical practice and economic benefits of the active fragment.

### SUMMARY

In order to solve the problem in the prior art that MIMP with an improvement effect on IBD easily produces immunogenicity and can hardly be prepared into a drug, the present disclosure provides an MP-B, a pharmaceutical preparation, and a use thereof. The MP-B of the present disclosure can significantly improve the pathologic colon shortening and colonic histopathologic score of IBD mice.

The present disclosure is implemented by the following technical solutions:

The present disclosure provides an MP-B, with an amino acid sequence shown in SEQ ID NO: 1.

Preferably, in the amino acid sequence shown in SEQ ID NO: 1, an amino acid Xaa at position 10 may be Arg, Phe, Glu, Thr, or absent, an amino acid Xaa at position 16 may be Asn, Val, Leu, Gly, or absent, and an amino acid Xaa at position 25 may be Ser, Glu, Met, Arg, or absent.

The present disclosure also provides a use of the MP-B described above in the preparation of an anti-IBD drug.

The present disclosure also provides a use of the MP-B described above in the preparation of an anti-IBD food or food additive.

The present disclosure also provides a use of the MP-B described above in the preparation of an anti-IBD health product.

Preferably, the use may refer to a use of the MP-B described above in the preparation of a drug for improving pathologic colon shortening of IBD.

Preferably, the use may refer to a use of the MP-B described above in the preparation of a drug for reducing a colonic histopathologic score of IBD.

The present disclosure also provides a pharmaceutical preparation, including the MP-B described above and a pharmaceutically acceptable carrier, excipient, or diluent.

Preferably, a dosage form of the pharmaceutical preparation may be an injection, a capsule, a tablet, a granule, a suspension, an enema, an emulsion, or a powder.

Compared with the prior art, the present disclosure has the following beneficial effects:

In the present disclosure, an acute IBD mouse model is established by a dextran sulfate sodium (DSS) chemical induction method, and the analysis means of colon morphology and histopathology are used to explore whether the MP-B shows an improvement effect on the IBD mouse model. Research results show that the intervention of the MP-B at the same dosage as MIMP significantly improves the colonic pathologic morphology in the IBD mouse model, reduces the colonic histopathologic score in the IBD mouse model, and shows the ability to interfere with the occurrence of IBD in mice. Compared with MIMP, the MP-B has a smaller molecular weight, which is beneficial to the drug preparation and application of MP-B. The present disclosure reveals the application potential of the MP-B in the preparation of an active product for preventing, intervening, and treating IBD.

Specific meanings of the abbreviations used in the present disclosure are as follows:

Thr: threonine; His: histidine; Val: valine; Gly: glycine; Ser: serine; Phe: phenylalanine; Asn: asparagine; Tyr: tyrosine; Ala: alanine; Leu: leucine; Glu: glutamic acid; Met: methionine; Pro: proline; Asp: aspartic acid; Arg: arginine; Lys: lysine; and Gln: glutamine.

### BRIEF DESCRIPTION OF THE DRAWINGS

FIG. 1 shows body weight change trends of mice in the model group and the blank control group, where compared with the blank control group, ^{#}P<0.05, ^{##}P<0.01, and ^{###}P<0.001; and the independent two-sample t-test is conducted for significance test;

FIG. 2 is a colon length comparison chart of mice in the blank control group, the model group, the MIMP positive control group, and the MP-B experimental group, where compared with the model group, *P<0.05, **P<0.01, and ***P<0.001; compared with the blank control group, ^{#}P<0.01; and one-way analysis of variance (ANOVA) is conducted for significance test; and

FIG. 3A shows histopathological micrographs of colons of mice in the blank control group, the model group, the MIMP positive control group, and the MP-B experimental group (HE staining 20×microscopy; A. blank control group, B. model group, C. MIMP positive control group, and D. MP-B experimental group); and

FIG. 3B shows a histopathologic score comparison chart (compared with the model group, *P<0.05, **P<0.01, and ***P<0.001; compared with the blank control group, ^{###}P<0.001; and one-way ANOVA is conducted for significance test).

### DETAILED DESCRIPTION OF THE EMBODIMENTS

The technical solutions in the examples of the present disclosure are clearly and completely described below with reference to the accompanying drawings in the examples of the present disclosure. Apparently, the described examples are merely a part rather than all of the examples of the present disclosure. The following description of at least one exemplary example is merely illustrative, and not intended to limit the present disclosure and application or use thereof in any way. All other examples obtained by a person of ordinary skill in the art based on the examples of the present disclosure without creative efforts shall fall within the protection scope of the present disclosure.

The reagents, materials, and devices used in the examples are shown in Table 1:

TABLE 1

| Name | Manufacturer |
|---|---|
| Male C57BL6 mice, clean grade | Shanghai Slack (Shanghai, China) |
| DSS | MP Biomedicals (CA, United States) |
| Phosphate-buffered saline (PBS) | Shanghai Boguang Biotechnology |
| | Co., Ltd. (Shanghai, China) |
| MIMP | Suzhou Qiangyao Biotechnology |
| | Co., Ltd. (Suzhou, China) |
| 4% Paraformaldehyde (PFA) | Shanghai Boguang Biotechnology |
| | Co., Ltd. (Shanghai, China) |

### EXAMPLE 1

### Experiment on an Intervention Effect of MP-B on DSS-Induced IBD in Mice

The MP-B used in this example had an amino acid sequence shown in SEQ ID NO: 1, in which an amino acid Xaa at position 10 was Glu, an amino acid Xaa at position 16 was Gly, and an amino acid Xaa at position 25 was Met, namely, SPLGSLDGRETMYNLGGVKYLFARMDQLKKQ.

### 1. Experimental Method

### 1.1 Establishment of an Acute IBD Mouse Model

The administration of a DSS solution with a specified concentration to mice can induce an acute IBD model characterized by diarrhea, hematochezia, ulcer, and granulocyte infiltration. Mice were randomly grouped according to body weights of the mice. 40 healthy male C57BL6 mice were divided into four groups each with 10 mice:

blank control group: the mice were each intragastrically administered with water every day at a volume of 0.4 mL/20 g;

model group: the mice were each intragastrically administered with a DSS aqueous solution of a mass fraction of 2.5 wt % consecutively for 7 days, where the DSS aqueous solution was freshly prepared and changed every day;

MIMP positive control group: the mice were each given a pre-administration process for one week, that is, the mice were each intragastrically administered with an MIMP solution of a mass fraction of 50 μg/kg for the first 7 days, and then from day 8, the mice were each intragastrically administered with a DSS aqueous solution of a mass fraction of 2.5 wt % (at a volume of 0.4 mL/20 g) and an MIMP solution of a mass fraction of 50 μg/kg (at a volume of 0.4 mL/20 g) every day; and

MP-B experimental group: the mice were each given a pre-administration process for one week, that is, the mice were each intragastrically administered with an MP-B solution of a mass fraction of 50 μg/kg for the first 7 days, and from day 8, the mice were each intragastrically administered with a DSS aqueous solution of a mass fraction of 2.5 wt % (at a volume of 0.4 mL/20 g) and an MP-B solution of a mass fraction of 50 μg/kg (at a volume of 0.4 mL/20 g) every day.

The body weight changes of mice in each group were recorded every day to determine whether the acute IBD mouse model was successfully established.

### 1.2 Sampling

Mice were each sacrificed by cervical dislocation and placed on an operating table, the abdominal cavity was exposed, and the intestinal conditions were observed to determine whether there was congestion, ulcer, and adhesion. A mouse colon between an anus end to an ileocecal end was integrally collected, and a length of the colon was measured; and the colon was dissected along a longitudinal axis, feces therein were rinsed off, and then the colon was stored in 4% paramethanol.

### 1.3 Histopathological Evaluation

The colon sample stored in 4% paramethanol in step 1.2 was subjected to histopathological section, stained with hematoxylin-eosin (HE), and dehydrated, obtained sections were sealed and examined under an optical microscope, and the histopathological scoring was conducted by two blind examination operators:

Scoring criteria: 0: no obvious inflammation; 1: moderate inflammatory infiltration in the basal layer; 2: moderate hyperplasia or severe inflammatory infiltration in the mucosa; 3: severe mucosal hyperplasia and absence of goblet cells; and 4: absence of crypt or ulcer.

### 1.4 Statistical Analysis

Experimental data in the above experimental method were expressed as (x̅±SD), the GraphPad Prism (ver. 8.0, GraphPad Software Inc., San Diego, Calif., USA) was used to plot a chart, the SPSS Program (ver. 25.0, SPSS Inc., Chicago, Ill., USA) was used for statistical test, and one-way ANOVA or independent two-sample t-test were used for significance test when the normality and homogeneity of variances were met. It was assumed that α=0.05, and P<0.05 indicates a statistically significant difference.

### 2. Experimental Results and Analysis

### 2.1 The MP-B Intervention Significantly Improved the Pathologic Colon Shortening of IBD Mice.

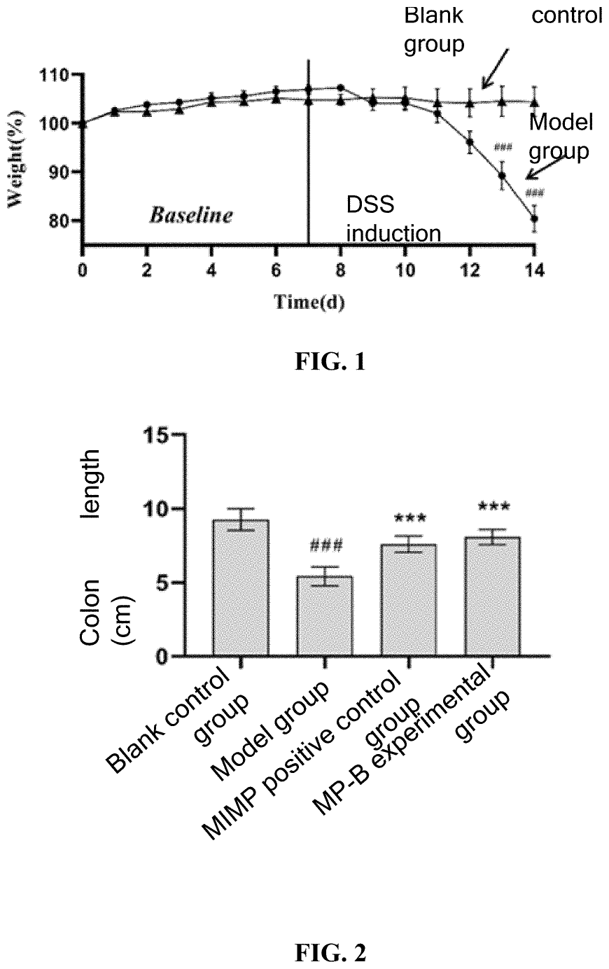

FIG. 1 shows weight change trends of mice in the model group and the blank control group, and it can be seen that, after one week of DSS induction, a body weight of mice in the model group decreased significantly (compared with the blank control group, ^{###}P<0.001, indicating a significant difference), indicating that the acute IBD mouse model was successfully established.

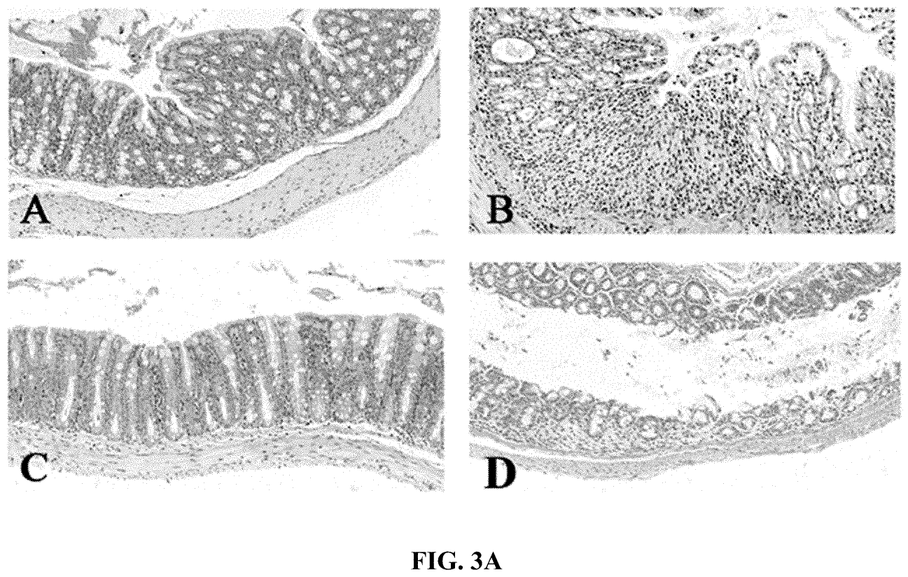

FIG. 2 is a colon length comparison chart of mice in the blank control group, the model group, the MIMP positive control group, and the MP-B experimental group, and it can be seen that the colon length (5.3±0.6) of mice in the model group was significantly smaller than the colon length (9.2±0.8) of mice in the blank control group (^{###}P<0.001), and the colon shortening of mice in the MP-B experimental group (colon length: 8.0±0.5) was significantly different from the colon shortening of mice in the model group (colon length: 5.3±0.6) (compared with the model group, ***P<0.001, indicating a significant difference), indicating that the MP-B intervention can significantly reverse this shortening with a comparable effect to the MIMP positive control group (colon length: 7.6±0.5), thereby improving the pathologic colon morphology of IBD mice.

### 2.2 The MP-B Intervention Significantly Reduced the Colonic Histopathologic Score of IBD Mice.

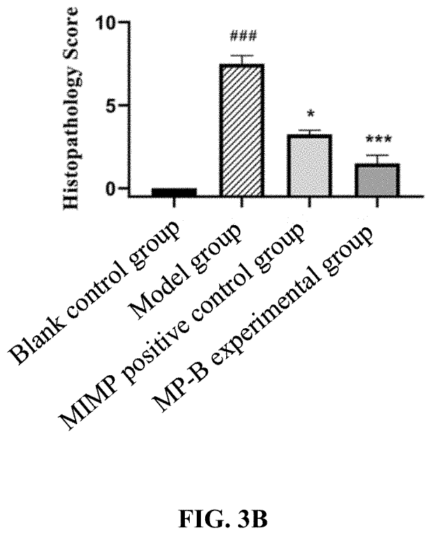

FIG. 3A shows histopathological micrographs of colons of mice in the blank control group, the model group, the MIMP positive control group, and the MP-B experimental group (HE staining 20×microscopy; A. blank control group, B. model group, C. MIMP positive control group, and D. MP-B experimental group); and FIG. 3B shows a histopathologic score comparison chart (compared with the model group, *P<0.05, **P<0.01, and ***P<0.001; compared with the blank control group, ^{###}P<0.001; and one-way ANOVA was conducted for significance test).

The histopathologic score was as follows: the blank control group: 0.0±0.0; the model group: 7.6±0.6; the MIMP positive control group: 3.1±0.2; and the MP-B experimental group: 1.4±0.6.

It can be seen from the colonic histopathologic score results that the MIMP intervention and the MP-B intervention both can significantly reduce the colonic histopathologic score of IBD mice (***P<0.001, indicating a significant difference). In the MP-B experimental group, the pathological conditions were improved accordingly, the mucosal epithelial structure was relatively complete, the morphology and structure of epithelial cells were normal, and there was no obvious inflammation, proving that the MP-B intervention can improve the large-area ulcer of the colonic mucosa induced by DSS, reduce the infiltration of lymphocytes and neutrophils, and further interfere with the occurrence of IBD.

### EXAMPLE 2

The reagents, materials, devices, and experimental method used in this example were the same as those in Example 1, except that the MP-B used in this example had an amino acid sequence shown in SEQ ID NO: 1, in which an amino acid Xaa at position 10 was Arg, an amino acid Xaa at position 16 was Asn, and an amino acid Xaa at position 25 was Glu, namely, SPLGSLDGRRTMYNLNGVKYLFAREDQLKKQ.

### EXAMPLE 3

The reagents, materials, devices, and experimental method used in this example were the same as those in Example 1, except that the MP-B used in this example had an amino acid sequence shown in SEQ ID NO: 1, in which an amino acid Xaa at position 10 was Thr, an amino acid Xaa at position 16 was Val, and an amino acid Xaa at position 25 was Ser, namely, SPLGSLDGRTTMYNLVGVKYLFARSDQLKKQ.

### EXAMPLE 4

The reagents, materials, devices, and experimental method used in this example were the same as those in Example 1, except that the MP-B used in this example had an amino acid sequence shown in SEQ ID NO: 1, in which an amino acid Xaa at position 10, an amino acid Xaa at position 16, and an amino acid Xaa at position 25 were each absent, namely, SPLGSLDGRTMYNLGVKYLFARDQLKKQ.

Examples 2 to 4 were tested according to the experimental method of Example 1, and analysis results were not much different from the results of Example 1, indicating that the intervention of the MP-B of the present disclosure can significantly improve the colonic pathologic morphology of the IBD mouse model and decrease the colonic histopathologic score of the IBD mouse model, and shows the ability to improve and interfere with the occurrence of IBD in mice.

The objectives, technical solutions, and beneficial effects of the present disclosure are further described in detail in the above specific examples. It should be understood that the above are merely specific examples of the present disclosure, but are not intended to limit the present disclosure. Any modification, equivalent replacement, or improvement made within the spirit and principle of the present disclosure shall fall within the protection scope of the present disclosure.

What is claimed is:

1. A polypeptide fragment B (MP-B), having an amino acid sequence set forth in SEQ ID NO: 1.

2. The MP-B according to claim 1, wherein in the amino acid sequence set forth in SEQ ID NO: 1,
an amino acid Xaa at position 10 is Arg, Phe, Glu, Thr, or absent;
an amino acid Xaa at position 16 is Asn, Val, Leu, Gly, or absent; and
an amino acid Xaa at position 25 is Ser, Glu, Met, Arg, or absent.

3. A method of treating inflammatory bowel disease (IBD), comprising administering the MP-B according to claim 1 in a preparation of an anti-IBD drug to a patient in need thereof.

4. A method of treating inflammatory bowel disease (IBD), comprising administering the MP-B according to claim 1 in a preparation of an anti-IBD food or food additive to a patient in need thereof.

5. A method of treating inflammatory bowel disease (IBD), comprising administering the MP-B according to claim 1 in a preparation of an anti-IBD health product to a patient in need thereof.

6. The method according to claim 3, wherein the anti-IBD drug is used for improving pathologic colon shortening of the IBD.

7. The method according to claim 3, wherein the anti-IBD drug is used for reducing a colonic histopathologic score of the IBD.

8. A pharmaceutical preparation, comprising the MP-B according to claim 1 and a pharmaceutically acceptable carrier, an excipient, or a diluent.

9. The pharmaceutical preparation according to claim 8, wherein a dosage form of the pharmaceutical preparation is an injection, a capsule, a tablet, a granule, a suspension, an enema, an emulsion, or a powder.


SEQUENCE LISTING (2 sequences)

SEQ ID NO: 1 - protein, 31 aa, Artificial Sequence
  Sequence is synthesized
  misc_feature (10)..(10): Xaa can be Arg, Phe, Glu, Thr, or absent
  misc_feature (16)..(16): Xaa can be Asn, Val, Leu, Gly, or absent
  misc_feature (25)..(25): Xaa can be Ser, Glu, Met, Arg, or absent
SPLGSLDGRXTMYNLXGVKYLFARXDQLKKQ

SEQ ID NO: 2 - protein, 61 aa, Lactobacillus plantarum
THTVGSYFSVQNGYVGAFSQALGNSEYAMNSPLGSLDGRTTMYNLLGVKYLFAREDQLKKQ